(19) United States
(12) Patent Application Publication
(10) Pub. No.: US 2026/0335089 A1
(43) Pub. Date: Oct. 29, 2026

(54) ORTHOPEDIC DEVICES AND METHODS OF USE

(71) Applicant: Scott Ortho Innovations, LLC, Spokane, WA (US)

(72) Inventors: David F. Scott, Spokane, WA (US); Robert J. Jones, Lago Vista, TX (US)

(73) Assignee: Scott Ortho Innovations, LLC, Spokane, WA (US)

(21) Appl. No.: 19/456,464

(22) Filed: Jan. 22, 2026

Related U.S. Application Data
(60) Provisional application No. 63/794,550, filed on Apr. 25, 2025.

(51) Int. Cl. A61F 2/30

(52) U.S. Cl. CPC A61F 2/30; A61F 2002/30649

(57) ABSTRACT

An example orthopedic device has a lengthwise axis and comprises an adapter, a main body, and an attachment member. The adapter has an adapter first end, an adapter second end, and an adapter recess. The adapter recess extends into the adapter from the adapter first end toward the adapter second end. The adapter recess has a first semi-spherical shape. The main body is configured to be partially disposed within the adapter recess. The main body has a first end, a second end, a central axis, and defines a protrusion. The protrusion is configured to be partially disposed within the adapter recess. The protrusion has a second semi-spherical shape allowing movement of the main body along more than one axis relative to the adapter when the main body is partially disposed within the adapter recess. The attachment member is configured to attach the main body to the adapter.

### FIELD

The disclosure relates to the field of medical devices. More specifically, the disclosure relates to orthopedic devices, particularly, joint replacement prosthetic devices.

### BACKGROUND

Not all human anatomy is the same; there is natural variability. As a result, the treatment applicable to one patient may not be appropriate for another. For example, each patient has a different bone structure and geometry. Therefore, properly positioning and fitting prosthetic devices to human bone is crucial for increasing patient satisfaction, improving health outcomes, and enhancing the durability and longevity of the device.

Trial prostheses are often used to fit prosthetic devices to respective portions of a patient's anatomy during surgical procedures. After shaping the patient's anatomy (e.g., femur, tibia), a surgeon temporarily fits trial devices instead of the actual final prosthesis. This enables the surgeon to test the fit of the prosthesis to the patient's anatomy. Multiple sizes and options are generally available, and the surgeon may evaluate more than one size or option to determine the best fit for the individual patient. This process continues until the most appropriate orthopedic device is selected. At the present time, the prosthetic device selected may not perfectly fit patient anatomy, and the orthopedic surgeon may have to make adjustments due to the limitations in device options. This may increase the operative time and complexity, increasing risks, complications, and cost, and may adversely affect the patient's outcomes, and/or reduce the function and longevity of the prosthetic device.

Therefore, a need exists for improved orthopedic devices and methods of use. The example orthopedic devices and methods described herein address that need.

### BRIEF SUMMARY OF SELECTED EXAMPLES

Various example orthopedic devices and methods of use are described.

An example orthopedic device has a lengthwise axis and comprises an adapter, a main body, and an attachment member. The adapter has an adapter first end, an adapter second end, and an adapter recess. The adapter recess extends into the adapter from the adapter first end toward the adapter second end. The adapter recess has a first semi-spherical shape. The main body is configured to be partially disposed within the adapter recess. The main body has a first end, a second end, a central axis, and defines a protrusion. The protrusion is configured to be partially disposed within the adapter recess. The protrusion has a second semi-spherical shape allowing movement of the main body along more than one axis relative to the adapter when the main body is partially disposed within the adapter recess. The attachment member is configured to attach the main body to the adapter.

Additional understanding of the inventive orthopedic devices and methods of use can be obtained by reviewing the detailed description of selected examples, below, and the referenced drawings.

### Description of Figures

FIG. 1 is a front view of an example orthopedic device.

FIG. 2 is a rear view of the orthopedic device illustrated in FIG. 1.

FIG. 3 is a side view of the orthopedic device illustrated in FIG. 1.

FIG. 4 is another side view of the orthopedic device illustrated in FIG. 1.

FIG. 5A is a bottom view of the orthopedic device illustrated in FIG. 1.

FIG. 5B is a cross-sectional view of the orthopedic device illustrated in FIG. 1 taken along line 5B-5B in FIG. 5A.

FIG. 6 is an exploded view of the orthopedic device illustrated in FIG. 1.

FIG. 7 is a front view of another example orthopedic device.

FIG. 8 is a rear view of the orthopedic device illustrated in FIG. 7.

FIG. 9 is a side view of the orthopedic device illustrated in FIG. 7.

FIG. 10 is another side view of the orthopedic device illustrated in FIG. 7.

FIG. 11A is a bottom view of the orthopedic device illustrated in FIG. 7.

FIG. 11B is a cross-sectional view of the orthopedic device illustrated in FIG. 7 taken along line 11B-11B in FIG. 11A.

FIG. 12 is an exploded view of the orthopedic device illustrated in FIG. 7.

FIG. 13 is a front view of another example orthopedic device.

FIG. 14 is a partial sectional view of the orthopedic device illustrated in FIG. 14.

FIG. 15 is a front view of another example orthopedic device.

FIG. 16 is a rear view of the orthopedic device illustrated in FIG. 15.

FIG. 17 is a side view of the orthopedic device illustrated in FIG. 15.

FIG. 18 is another side view of the orthopedic device illustrated in FIG. 15.

FIG. 19A is a bottom view of the orthopedic device illustrated in FIG. 15.

FIG. 19B is a cross-sectional view of the orthopedic device illustrated in FIG. 15 taken along line 19B-19B in FIG. 19A.

FIG. 20 is a front view of another example orthopedic device.

FIG. 21. is a partial front sectional view of the orthopedic device illustrated in FIG. 20.

FIG. 22 is a side view of the orthopedic device illustrated in FIG. 20.

FIG. 23 is a partial side sectional view of the orthopedic device illustrated in FIG. 20.

FIG. 24 is an exploded view of the orthopedic device illustrated in FIG. 20.

FIG. 25 is a sectional view of another example orthopedic device.

FIG. 26 is an exploded view of the orthopedic device illustrated in FIG. 25.

FIG. 27 is a front sectional view of another example orthopedic device.

FIG. 28 is a schematic of an example method of using an orthopedic device.

### DETAILED DESCRIPTION OF SELECTED EXAMPLES

The following detailed description and the appended drawings describe and illustrate various example orthopedic devices and methods of use. The description and illustration of these examples are provided to enable one skilled in the art to make and use an orthopedic device and practice associated methods of use. They do not limit the scope of the claims in any manner. The invention is capable of being practiced or carried out in various ways and the examples described and illustrated herein are merely selected examples of the various ways of practicing or carrying out the invention and are not exhaustive.

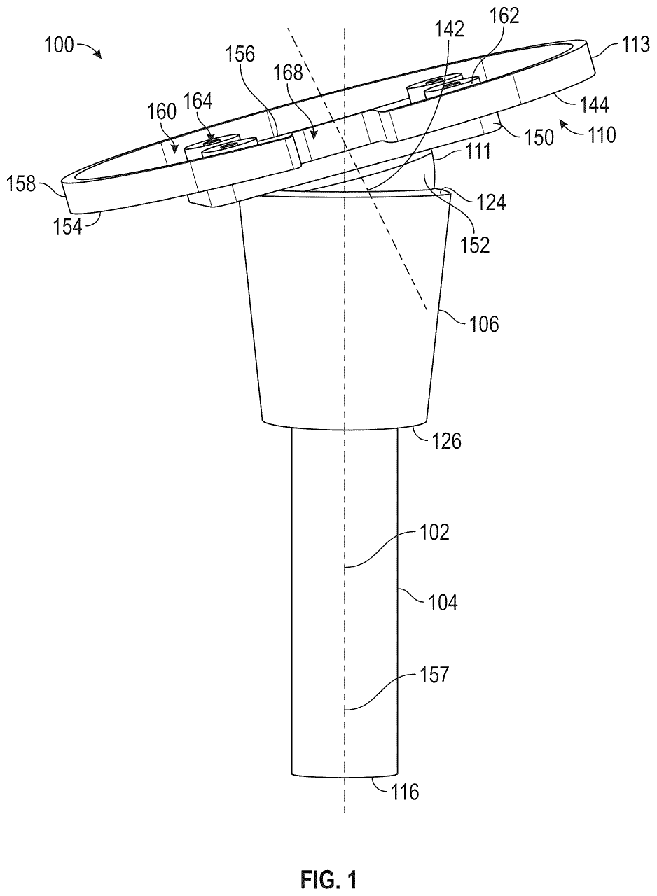

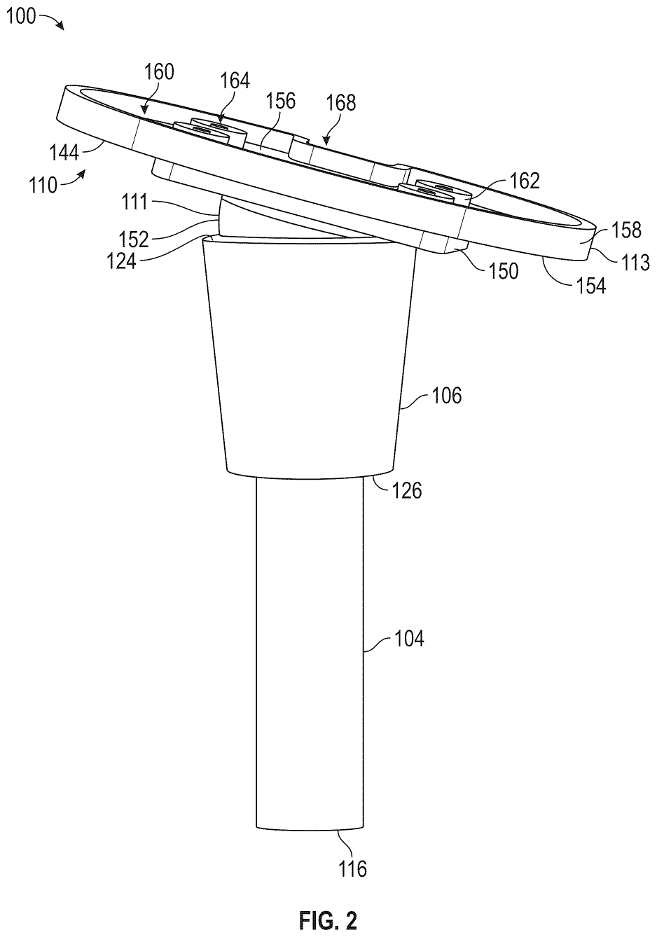

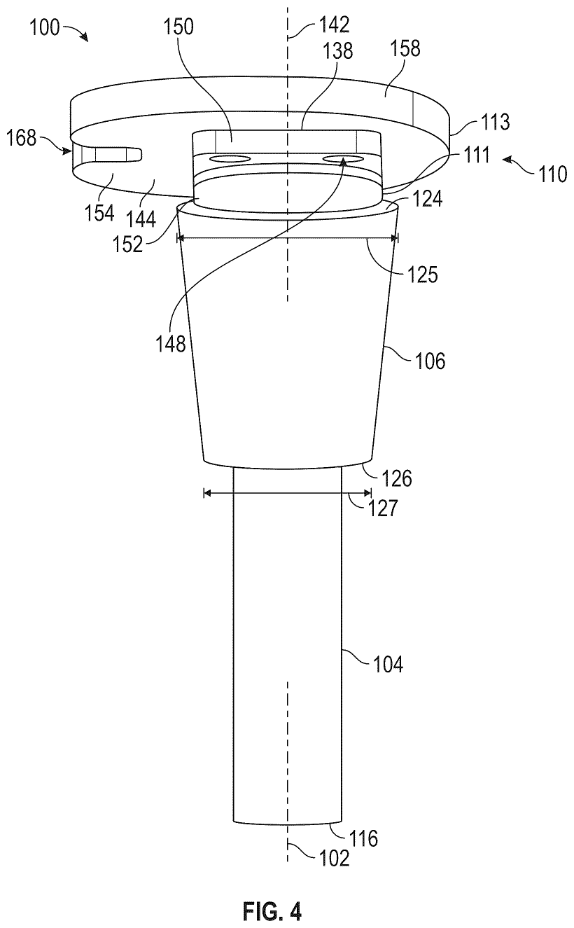

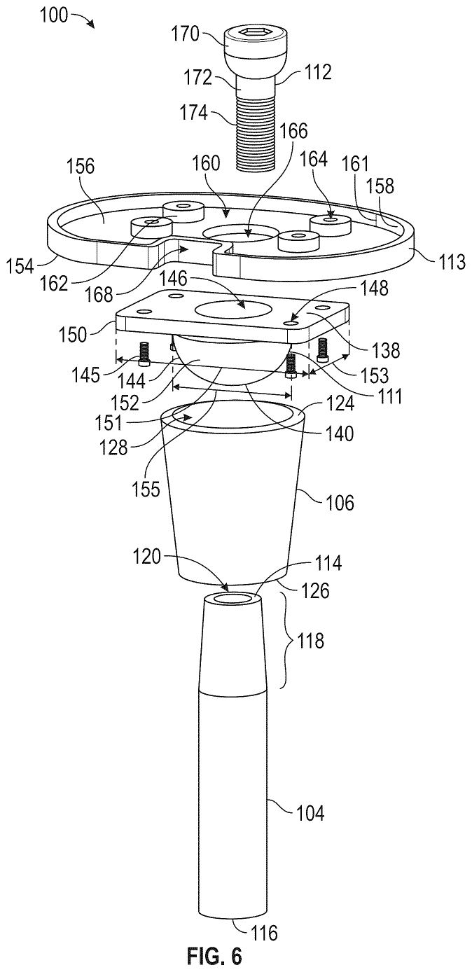

FIGS. 1 through 6 illustrate an example orthopedic device 100. In the illustrated embodiment, the orthopedic device 100 is a portion of a knee prosthesis (e.g., proximal tibial knee replacement). However, an orthopedic device that includes the features and components described herein can comprise any suitable orthopedic device and be used relative to any suitable patient anatomy. For example, the features and components described herein can be used to replace a second femur in a knee replacement, and in other joints within a patient's anatomy.

In the illustrated embodiment, the orthopedic device 100 has a lengthwise axis 102, a stem 104, an adapter 106, a main body 110, and an attachment member 112.

The stem 104 has a stem first end 114, a stem second end 116, a stem length 117, a stem tapered portion 118, a stem recess 120, and a stem recess thread 122. The stem first end 114 is disposed within the adapter 106. The stem length 117 extends from the stem first end 114 to the stem second end 116. In the embodiment shown, the stem length 117 is about 65 mm. However, in alternative embodiments, a stem can have any suitable length, such as those less than, greater than, or equal to 65 mm, or having a range equal to or exceeding greater than, or less than, 10 mm to 600 mm.

The stem tapered portion 118 extends from the stem first end 114 toward the stem second end 116. In the illustrated embodiment, the stem tapered portion 118 is disposed within the adapter 106 and is about 25 mm. However, in alternative embodiments, a stem tapered portion can be any suitable length, such as those less than 60 mm, or greater than 2 mm. The stem recess 120 extends from the stem first end 114 toward the stem second end 116 and receives a portion of the attachment member 112. The stem thread 122 is defined within the stem recess 120 and mates with the attachment member thread 174 of the attachment member 112. In the embodiment shown, the stem 104 is an elongate member that extends from the stem first end 114 to the stem second end 116. Although this embodiment of the orthopedic device illustrates the stem 104, alternative embodiments can omit the inclusion of a stem and/or can include an adapter having a different external shape and/or other design features. For example, an adapter can comprise, consist of, or include a keel or a plurality of keels.

The adapter 106 has an adapter first end 124, an adapter second end 126, an adapter first end recess 128, an adapter second end recess 130, and an adapter passageway 132. In the illustrated embodiment, the adapter 106 is a conical frustum. The adapter first end 124 has a first width 125 measured orthogonally to the lengthwise axis 102 and the adapter second end 126 has a second width 127 measured orthogonally to the lengthwise axis 102. The second width 127 is less than the first width 125. However, in alternative embodiments a second width can be greater than a first width. The adapter 106 tapers from the adapter first end 124 to the adapter second end 126. However, in alternative embodiments, an adapter can omit a taper, taper only along a portion of its length, or taper in the opposite direction. In even further embodiments, an adapter can include a keel, a plurality of keels, an external shape alternative to that shown, and/or have other design features.

The adapter first end recess 128 extends into the adapter 106 from the adapter first end 124 and toward the adapter second end 126. The adapter first end recess 128 has a first semi-spherical shape and receives a portion of the main body 110 (e.g., first portion 111). The adapter second end recess 130 extends into the adapter 106 from the adapter second end 126 and toward the adapter first end 124. The adapter second end recess 130 tapers from the adapter second end 126 toward the adapter first end 124 and receives the stem tapered portion 118. Although the illustrated embodiment depicts the entire stem tapered portion 118 disposed within the adapter second end recess 130 when the orthopedic device 100 is assembled, in alternative embodiments only a portion of a stem, or a stem tapered portion, can be disposed within a second end recess. The adapter passageway 132 extends from the adapter first end recess 128 to the adapter second end recess 130 and receives a portion of the attachment member 112.

In the illustrated embodiment, the main body 110 comprises a first portion 111 and a second portion 113. Therefore, any feature and/or component of a first portion 111 and/or a second portion 113 described herein is a part of the main body 110 and these features and components should be considered features and components of a main body. The main body 110 is partially disposed within the adapter 106 (e.g., the adapter first end recess 128). In the illustrated embodiment, the second portion 113 is a plate 144. However, in alternative embodiments, the second portion can be any suitable structure. In even further embodiments, a second portion can be omitted.

The first portion 111 has a first end 138, a second end 140, a central axis 142, and defines a passageway 146, a plurality of openings 148, an elongate portion 150, and a protrusion 152. The first portion 111 is partially disposed within the adapter first end recess 128. The central axis 142 is independent from, and moveable relative to, the lengthwise axis 102.

The passageway 146 extends through the elongate portion 150 and the protrusion 152 and from the first end 138 to the second end 140. The passageway 146 has a first portion 147 and a second portion 149. The first portion 147 defines a semi-spherical shape that receives the attachment member head 170 and allows movement between the main body 110 (e.g., the first portion 111) and the attachment member 112. The second portion 149 defines a conical taper that tapers from the second end 140 to the first portion 147 and allows movement between the main body 110 (e.g., the first portion 111) and the attachment member 112. Each opening of the plurality of openings 148 extends through the elongate portion 150 and receives a fastener 145 which attaches the first portion 111 to the second portion 113. In the illustrated embodiment, the first portion 111 is attached to the second portion 113 by a plurality of fasteners 145 each of which extends through an opening of the plurality of openings 148 and into the second portion 113. However, in alternative embodiments, a first portion can be attached to a second portion by any suitable means such as, but not limited to, an adhesive, or a first portion and a second portion can comprise a single, unitary component.

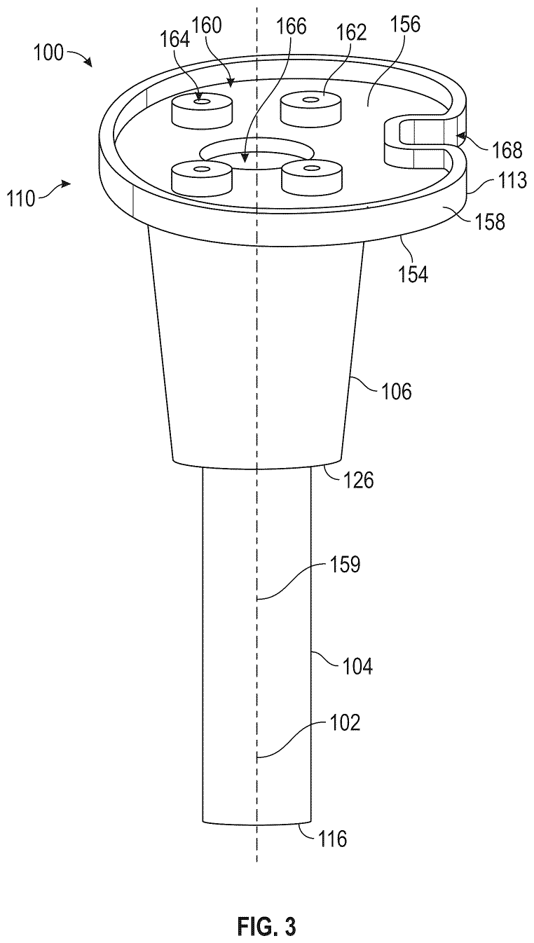

In the embodiment shown, the elongate portion 150 extends from the first end 138 toward the second end 140, is substantially planar, and has an elongate portion length 151 and an elongate portion width 153. The protrusion 152 extends from the elongate portion 150 to the second end 140. The protrusion 152 is partially disposed within the adapter first end recess 128, has a width 155, and has a second semi-spherical shape that mates with the first semi-spherical shape defined by the adapter 106. The width 155 of the protrusion 152 is less than the elongate portion length 151 and the elongate portion width 153. The first semi-spherical shape and the second semi spherical shape can have the same radius of curvature. However, in alternative embodiments, a radius of curvature of a first semi-spherical shape and a second semi-spherical shape can be slightly different to allow one to fit in the other better. This structural arrangement allows movement of the main body 110 (e.g., the first portion 111 and as a result of its attachment to the first portion 111 movement of the second portion 113) along more than one plane relative to the adapter 106 and about the lengthwise axis 102. For example, as shown in FIGS. 1 and 3, the main body 110 (e.g., the first portion 111 and the second portion 113) are moveable relative to a first plane 157 and a second plane 159 and about the lengthwise axis 102. As a result, the central axis 142 of the main body 110 can be disposed independently at any suitable angle relative to a first plane 157 and a second plane 159 allowing for customized placement of the main body relative to the adapter 106, thus relative to any extensions of the adapter 106, allowing for a unique fit to any patient's unique anatomy. This customized placement of the main body 110 relative to the adapter 106 is beneficial at least because it accommodates the unique anatomical composition of each patient and the various angles between bones and joints, which can vary from person to person. For example, the angular orientation of a first tibial plateau joint surface of the knee relative to a long axis of the tibial diaphyseal shaft is unique for each patient. The embodiments described herein allow a surgeon implanting an orthopedic device in the first tibia to adjust the device to match perfectly each patient's unique personalized anatomy. Therefore, the variability in the orthopedic devices described herein allows for adjustments to account for the differences between individual anatomies. This is accomplished by allowing adjustability in all planes independently due to the spherical configuration of various components described herein.

In the illustrated embodiment, the second portion 113 has a second side 154, a first side 156, a wall 158, a recess 160, a plurality of protrusions 162, a plurality of openings 164, a passageway 166, and a notch 168. Although the second portion 113 is illustrated as a separate component from the first portion 111, in alternative embodiments, a first portion and a second portion can comprise a single, unitary piece.

In the illustrated embodiment, the first portion 111 is attached to the second side 154 of the second portion 113. The first side 156 is opposably facing the second side 154 and is surrounded by the wall 158. The wall 158 extends from the first side 156 and has a height 161 that is about 6 mm. However, a wall can have any suitable height including, but not limited to, those less than 30 mm, or greater than 1 mm. The recess 160 extends from the wall 158 and into the second portion 113 to the first side 156. As a result, the recess 160 is defined by the first side 156 and the wall 158.

Each protrusion of the plurality of protrusions 162 extends from the first side 156 and away from the second side 154. In the illustrated embodiment, the plurality of protrusions 162 comprise four protrusions. However, any suitable number of protrusions can be included on a main body (e.g., a second portion), such as zero, one, more than one, two, three, five, more than five, and any other number considered suitable for a particular embodiment. Each opening 164 of the plurality of openings 164 extends through a distinct protrusion of the plurality of protrusions 162 and receives a fastener 145. The plurality of fasteners 145 secure the first portion 111 to the second portion 113 and comprise threaded members.

The passageway 166 is coaxial with, and aligned with, the passageway 146 of the first portion 111 and extends from the second side 154 to the first side 156 (e.g., the recess 160). The notch 168 is defined by the second side 154, the first side 156, and the wall 158.

In the illustrated embodiment, the attachment member 112 has an attachment member head 170, an attachment member elongate portion 172, and an attachment member thread 174. The attachment member 112 attaches the main body 110 (e.g., the first portion 111, and the second portion 113 as a result of its attachment to the first portion 111) to the adapter 106. In the embodiment shown, the attachment member 112 is a screw. For example, the screw can be a polyaxial screw that allows a main body to include a multitude of variable angles relative to an adapter. However, in alternative embodiments any suitable type of attachment member can be used to attach a main body (e.g., a first portion) to an adapter. For example, an attachment member can include, but is not limited to, a bolt, screw, rivet, anchor, nail, insert, stud, any other structure considered suitable for a particular embodiment, and any suitable structure with a helical thread. Any type of attachment member that can allow a main body to include a multitude of variable angles relative to an adapter may be used. While some components described herein have been described as being attached to one another, or one component has been described as being disposed in another component, any component described herein can be adapted to be, or configured to be, attached to another component, or disposed in another component, and/or can be provided in a kit for use.

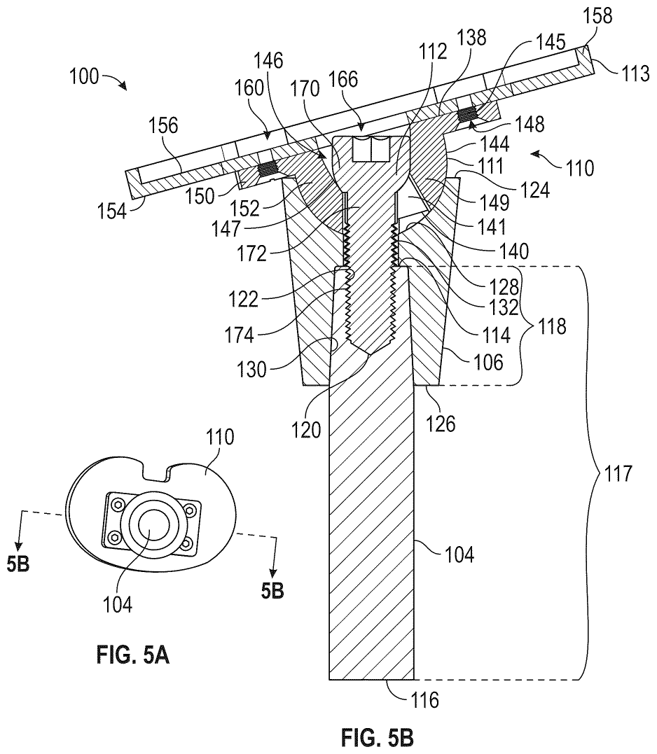

The attachment member 112 is disposed through the passageway 166 and the passageway 146 such that the attachment member head 170 is disposed within the first portion 147 of the passageway 146. The attachment member elongate portion 172 is disposed within the second portion 149 of the passageway 146, extends through the adapter passageway 132, and into the stem recess 120. The attachment member thread 174 extends along the circumference of the attachment member elongate portion 172 and mates with the stem recess thread 122 such that the attachment member 112 is attached to the stem 104. In the illustrated embodiment, the attachment member 112 is an M8 polyaxial screw. However, an attachment member can comprise any suitable component capable of attaching a main body (e.g., a first portion, a second portion) to a stem or other component described herein, including M1 screws, M2 screws, M3 screws, M4 screws, M5 screws, M6 screws, M7 screws, M8 screws, M9 screws, M10 screws, polyaxial screws, and any other component considered suitable for a particular embodiment. The type of attachment member used can depend on the anatomical place in which an orthopedic device is being utilized. For example, an orthopedic device that is to be implanted in a finger could potentially require a smaller attachment member than an orthopedic device that is to be implanted in a leg. As shown in FIG. 5B, the main body 110 is moveable relative to the attachment member 112 as a result of the structural configuration of the main body 110 (e.g., the inclusion of the first portion 147 and the second portion 149 of the recess 146).

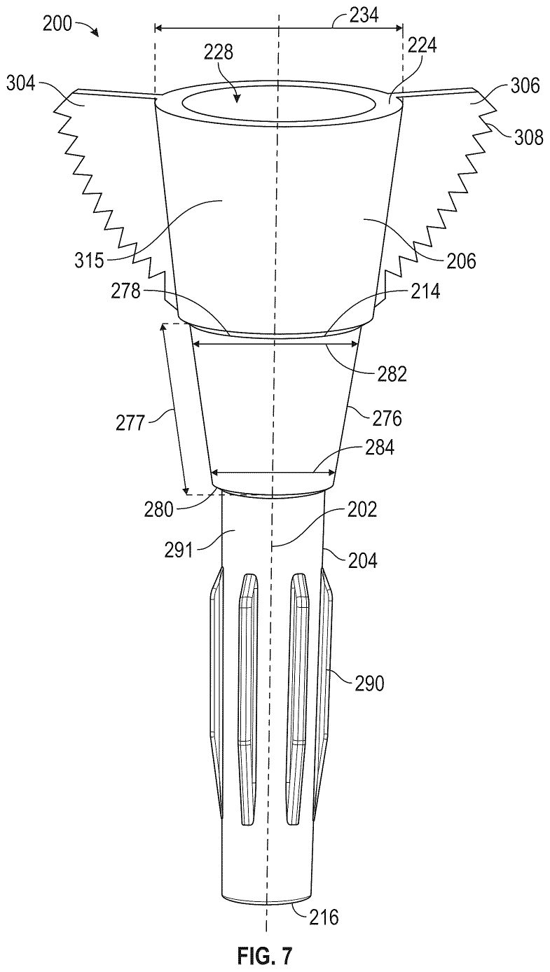

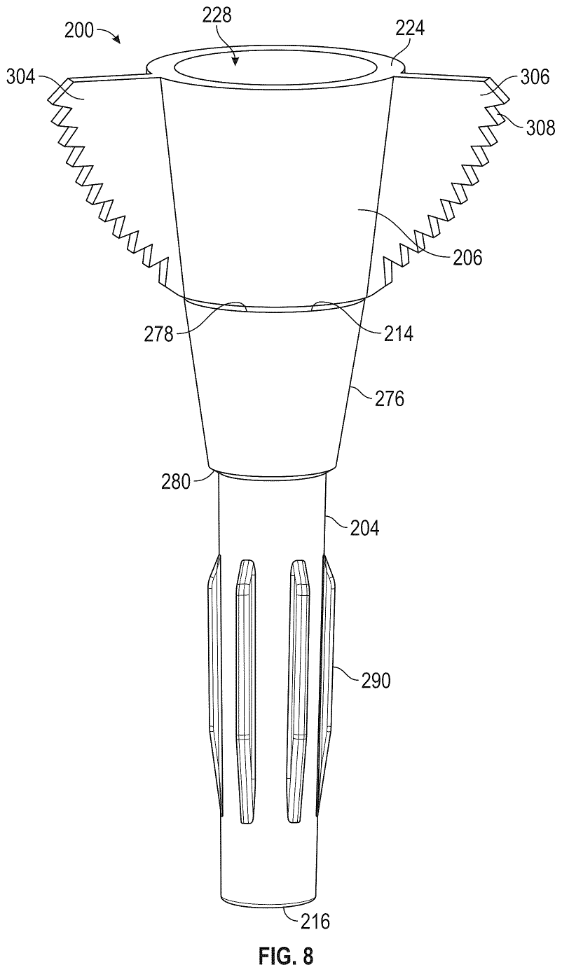

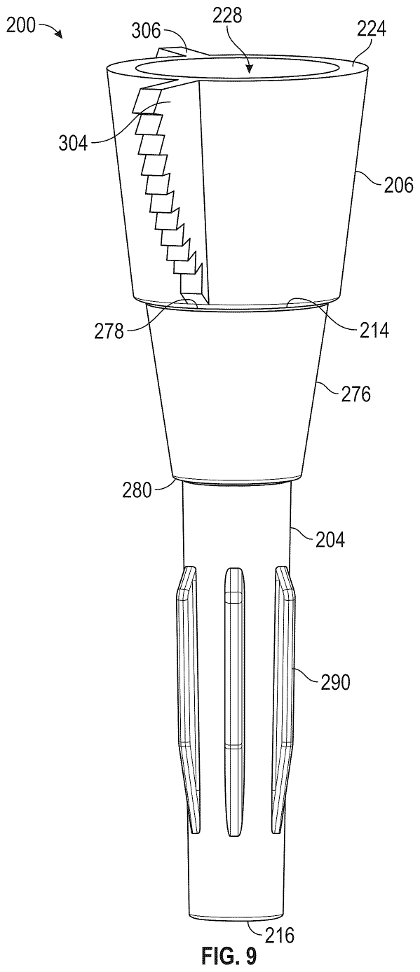

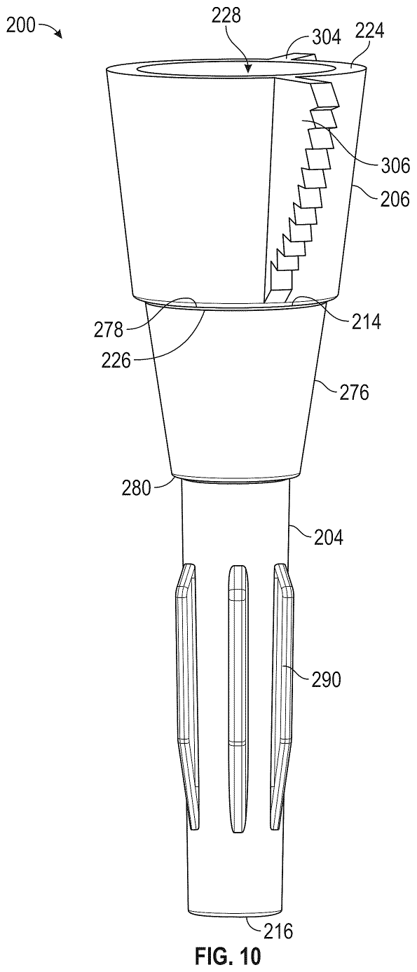

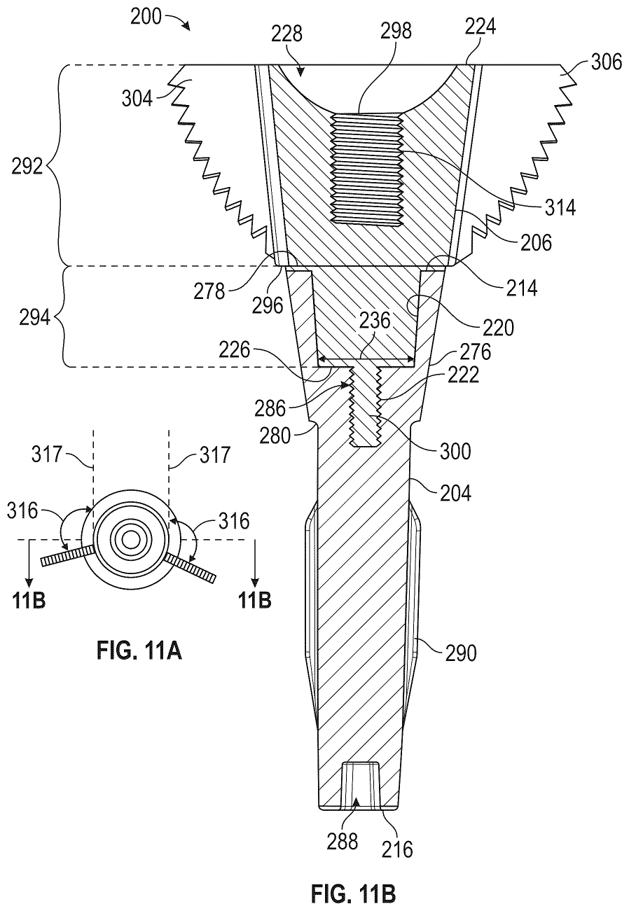

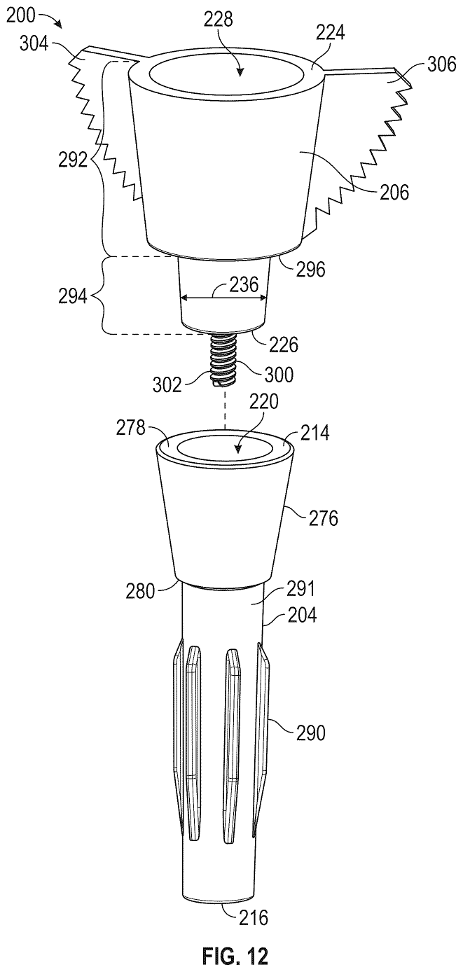

FIGS. 7 through 12 illustrate another example orthopedic device 200. The orthopedic device 200 is similar to the orthopedic device 100 illustrated in FIGS. 1 through 6 and described above, except as detailed below.

In the illustrated embodiment, the orthopedic device 200 has a lengthwise axis 202, a stem 204, and an adapter 206. While not illustrated, orthopedic device 200 can include a main body (e.g., a first portion, a second portion) and an attachment member.

In the illustrated embodiment, the stem 204 has a stem first end 214, a stem second end 216, a stem recess 220, a stem recess thread 222, a stem flange 276, a stem flange first end 278, a stem flange second end 280, a first flange width 282, a second flange width 284, a second stem recess 286, a stem second end recess 288, a plurality of stem protrusions 290, and an exterior surface 291.

The stem 204 tapers from the stem first end 214 toward the stem second end 216. The stem recess 220 extends from the stem first end 214 toward the stem second end 216 and receives a portion of the adapter 206. The second stem recess 286 extends from the stem recess 220 towards the stem second end 216 and receives a portion of the adapter 206. The stem recess thread 222 is defined within the second stem recess 220 and mates with the adapter thread 302 of the adapter attachment member 300.

The first flange width 282 is defined at the stem first end 214 and the second flange width 284 is defined between the stem first end 214 and the stem second end 216. The second flange width 284 is less than the first flange width 282. In the illustrated embodiment, the stem flange 276 has a length 277 that is about 20 mm. However, in alternative embodiments, a stem flange can have any suitable dimensions including, but not limited to, those between about 2 mm to about 60 mm. Use of the term “about” herein allows for 10% deviation from the value described.

The stem second end recess 288 extends from the stem second end 216 toward the stem first end 214. The stem second end recess 288 provides structure that allows a hex driver, or any other suitable tool, access to tighten the orthopedic device 200 into a locked position.

Each stem protrusion of the plurality of stem protrusions 290 extends along a portion of the exterior surface 291 of the stem 204 between the stem flange 276 and the stem second end 216. Each stem protrusion of the plurality of stem protrusions 290 extends from the exterior surface 291 and away from the lengthwise axis 202. Each protrusion of the plurality of stem protrusions 290 is equally spaced from an adjacent protrusion of the plurality of stem protrusions 290 about the stem 204. However, in alternative embodiments, a plurality of stem protrusions can have varying spacing between adjacent protrusions. In the embodiment shown, the plurality of stem protrusions comprise six stem protrusions. However, in alternative embodiment, any suitable number of stem protrusions can be included on a stem, such as zero, one, more than one, two, a plurality, three, four, five, six, more than six, and any other number considered suitable for a particular embodiment.

In the illustrated embodiment, the adapter 206 has an adapter first end 224, an adapter second end 226, an adapter recess 228, an adapter first portion 292, an adapter second portion 294, an adapter shoulder 296, a second adapter recess 298, an adapter attachment member 300, an adapter thread 302, a first keel 304, a second keel 306, and a plurality of keel barbs 308.

The adapter 206 is attached to the stem 204 and extends from the adapter first end 224 to the adapter second end 226. The adapter first portion 292 extends from the adapter first end 224 toward the adapter second end 226 and to the adapter shoulder 296. The adapter second portion 294 extends from the adapter second end 226 toward the adapter first end 224 and to the adapter shoulder 296. The adapter second portion 294 is disposed in the stem recess 220.

In the illustrated embodiment, the adapter first end 224 has a first width 234 measured orthogonally to the lengthwise axis 202 and the adapter second end 226 has a second width 236 measured orthogonally to the lengthwise axis 202. The second width 236 is less than the first width 234. The adapter first portion 292 tapers from the adapter first end 224 to the adapter shoulder 296. The adapter second portion 294 tapers from the adapter shoulder 296 to the adapter second end 226.

The adapter recess 228 extends into the adapter 206 from the adapter first end 224 toward the adapter second end 226. The second adapter recess 298 extends from the adapter recess 228 towards the adapter second end 226. The adapter 206 defines a thread 314 within the second adapter recess 298 that is sized to mate with a thread of an attachment member such that attachment between a main body (e.g., a first portion) and the adapter 206 can be achieved.

The adapter attachment member 300 extends from the adapter second end 226, away from the adapter first end 224, and defines the adapter thread 302. The adapter attachment member 300 is disposed in the second stem recess 286, and the adapter thread 302 mates with the stem recess thread 222 such that attachment between the adapter 206 and the stem 204 can be accomplished.

In the illustrated embodiment, the first keel 304 and the second keel 306 are attached to the adapter first portion 292 and extend away from the lengthwise axis 202. Although the illustrated embodiment depicts only two keels, any suitable number of keels may be attached to an adapter including, but not limited to, one, more than one, two, a plurality, three, more than three, and any other number considered suitable for a particular embodiment. In the embodiment shown, each of the first keel 304 and the second keel 306 extends from the adapter 206, away from the lengthwise axis 202, and away from a front side 315 of the adapter 206. Each of the first keel 304 and the second keel 306 is disposed at an angle 316 relative to lines 317 that are tangent to the first portion 292 of the adapter 206 that is greater than 90 degrees and less than 180 degrees. In the illustrated embodiment, the angle 316 is approximately 110 degrees. However, alternative embodiments can include any suitable angle. Each of the first keel 304 and the second keel 306 prevent rotation of the orthopedic device 200 subsequent to implantation.

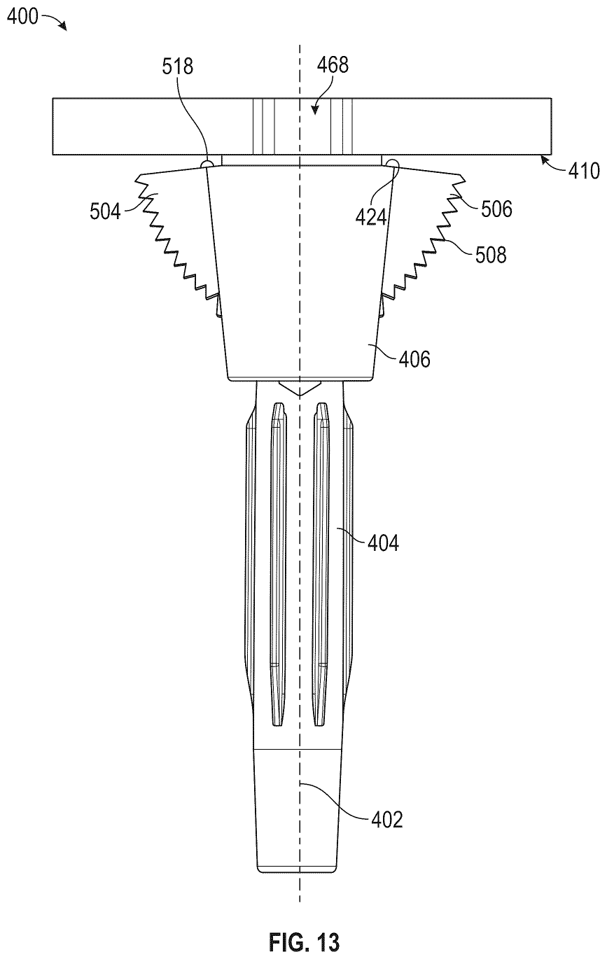

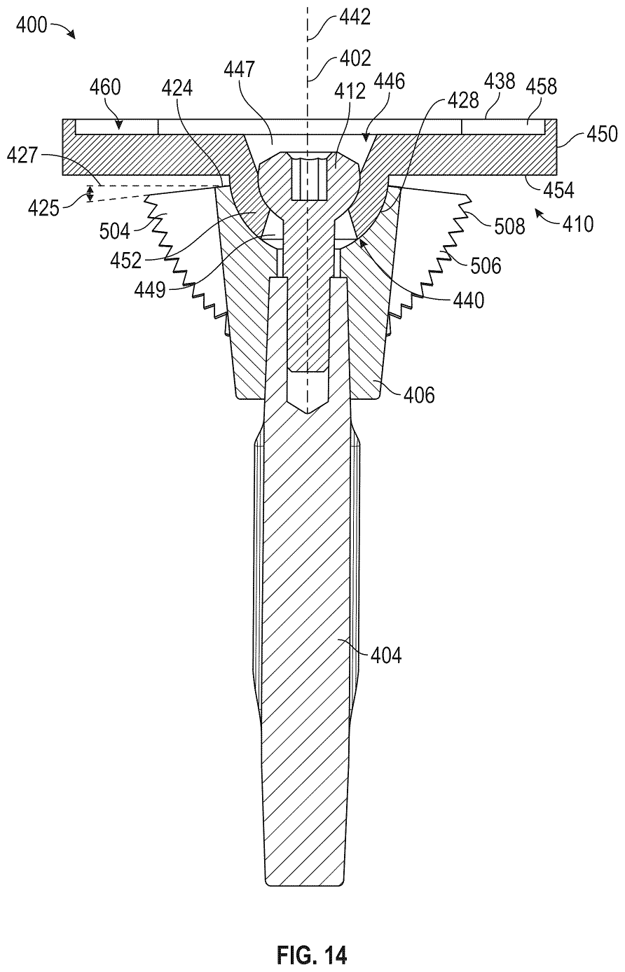

FIGS. 13 and 14 illustrate another example orthopedic device 400. The orthopedic device 400 is similar to the orthopedic device 100 illustrated in FIGS. 1 through 6 and described above, except as detailed below.

In the illustrated embodiment, the orthopedic device 400 has a lengthwise axis 402, a stem 404, an adapter 406, a main body 410, and an attachment member 412.

The adapter first end 424 is disposed at an angle 425 relative to a plane 427 that is orthogonal to the lengthwise axis 402 that is equal to about 7.5 degrees. However, an angle can be between about 3 degrees and about 30 degrees, be greater than, or less than, 30 degrees, and any other angle considered suitable for a particular embodiment. Any angle that allows the main body to rotate to its full excursion extension of an embodiment and movement of the main body is not impeded is considered suitable.

The adapter has a first keel 504 and a second keel 506. Each of the first keel 504 and the second keel 506 are attached to the adapter 406 and extend away from the lengthwise axis 402. In the embodiment shown, each of the first keel 504 and the second keel 506 are disposed at an angle 518 relative to the adapter first end 424. In the illustrated embodiment, the angle 518 is about 180 degrees. However, in alternative embodiments, an angle at which a keel is disposed relative to an adapter first end can be any suitable angle including, but not limited to, angles less than 180 degrees, angles greater than 180 degrees, and any other angle considered suitable for a particular embodiment. The structural arrangement of the adapter first end 424, the first keel 504, and the second keel 506 (e.g., angles 425, 518) allows the main body 410 to have a broader range of movement relative to devices that have adapter first ends and/or keels that are disposed on planes that are orthogonal to a lengthwise axis of a prosthetic device.

In the illustrated embodiment, the main body 410 comprises a single, unitary component that combines the features of the first portion 147 and the second portion 149, such as those described herein. Thus, the main body 410 has a first end 438, a second end 440, a central axis 442, a passageway 446, an elongate portion 450, a protrusion 452, a wall 458, a recess 460, and a notch 468. The second end 440 (e.g., protrusion 452) is illustrated as being a spherical male portion of a spherical modular junction. However, in alternative embodiments, a stem first end may include a protrusion (e.g., be a spherical male portion of the spherical modular junction). Further, a main body (e.g., protrusion) can be formed from any suitable material including cobalt-chromium, titanium, stainless steel, ceramics, medical-grade polymers, and any other material considered suitable for a particular embodiment. Cobalt-chromium is beneficial at least because it is a strong metal that can be used in implants, can be machined and polished to ensure that the spherical modular junction is able to move to accommodate all different patient anatomies. Further, in alternative embodiments, a main body and/or adapter may be coated with a material that increases friction between the main body and/or adaptor and a component being attached to the main body and/or adaptor (e.g., a protrusion and an adapter within a first end recess). Examples of materials considered suitable to coat a component of an orthopedic device include, but are not limited to, a ceramic or diamond coating. The inclusion of a coating on a component of an orthopedic device reduces slippage between components (e.g., a protrusion moving relative to an adapter). Alternative to, or in combination with, a coating, a main body and/or adapter may also have micro and/or macro textures on a surface thereof. For example, a surface of a main body and/or adapter may have a pattern such as a surface with dimples and/or facets such as a geodesic dome or a cut diamond thereon. The passageway 446 extends through the elongate portion 450 and the protrusion 452 and from the first end 438 to the second end 440. The passageway 446 has a first portion 447 and a second portion 449, as described herein. The protrusion 452 extends from the elongate portion 450 and is disposed within an adapter first end recess 428.

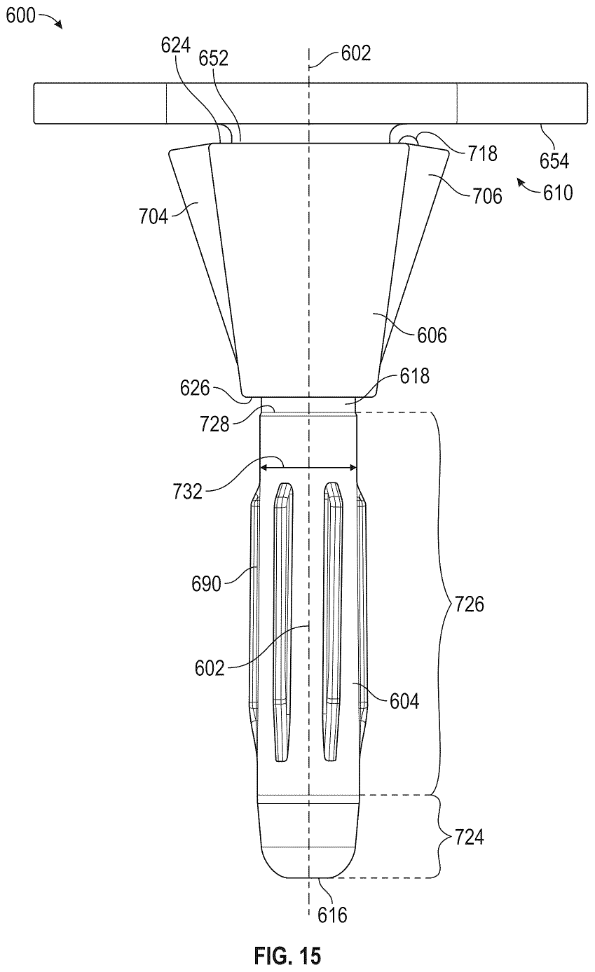

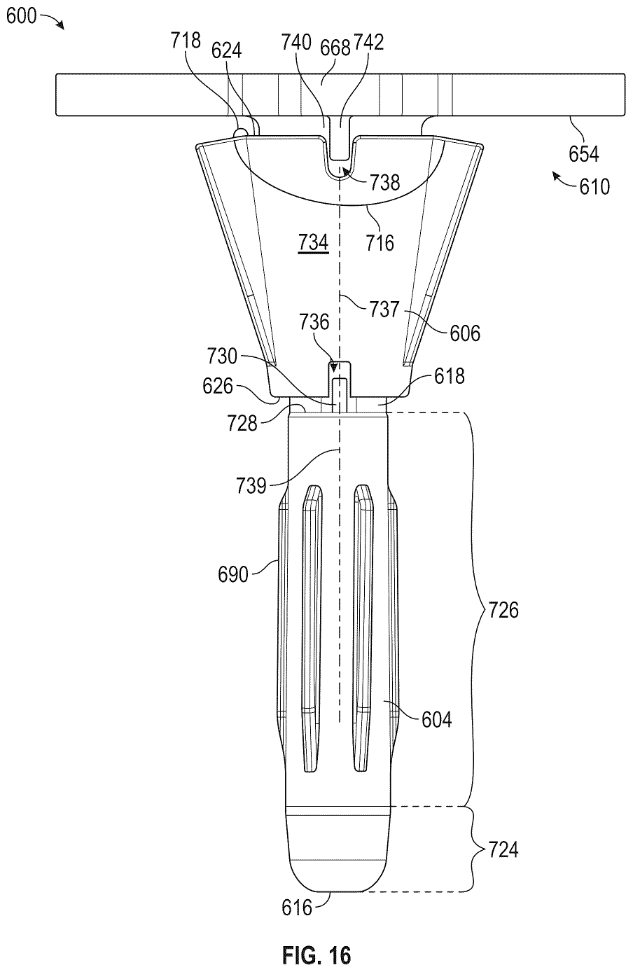

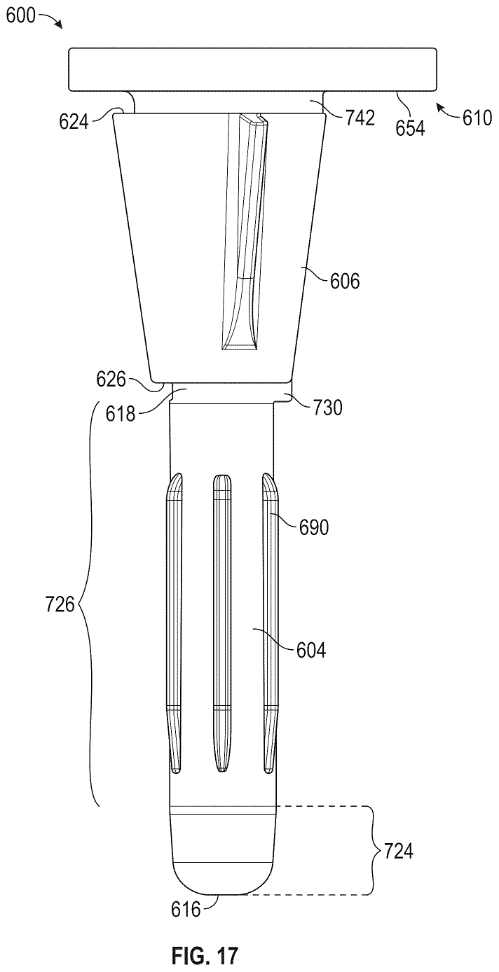

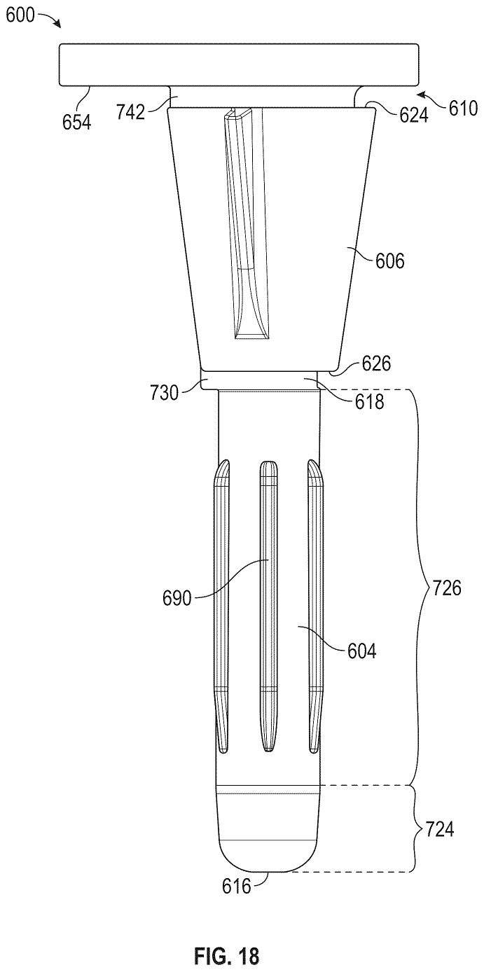

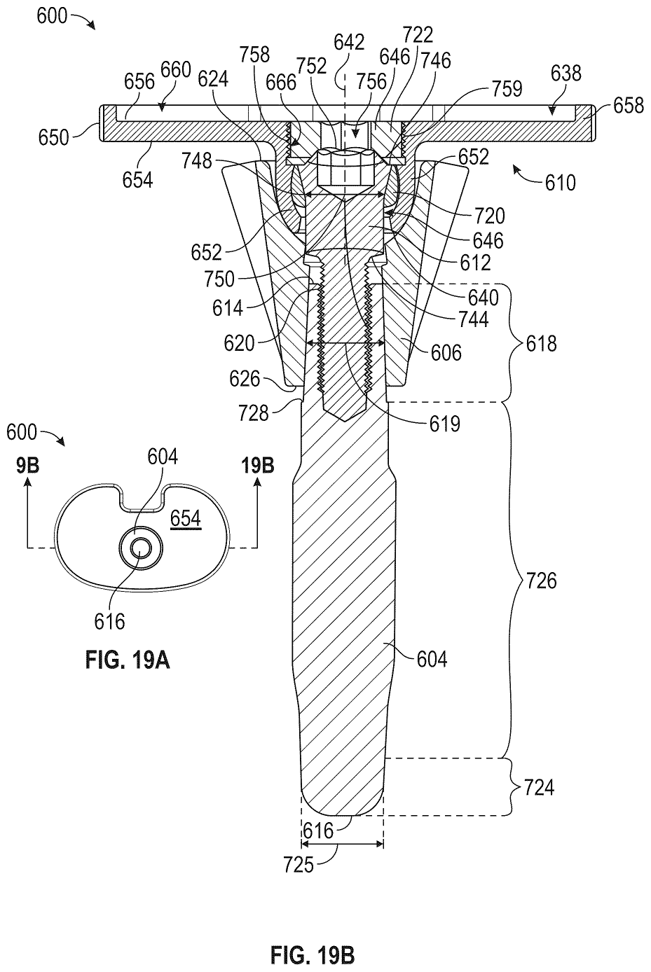

FIGS. 15 through 19b illustrate another example orthopedic device 600. The orthopedic device 600 is similar to the orthopedic devices described above, except as detailed below.

In the illustrated embodiment, the orthopedic device 600 has a lengthwise axis 602, a stem 604, an adapter 606, a main body 610, an attachment member 612, a compression ring 720, and a compression plug 722.

In the illustrated embodiment, the stem 604 has a stem first end 614, a stem second end 616, a stem tapered portion 618, a plurality of stem protrusions 690, a stem second portion 724, a stem intermediate portion 726, a stem intermediate portion shoulder 728, and a stem projection 730.

The stem second portion 724 extends from the stem second end 616 to the stem intermediate portion 726. The stem intermediate portion 726 extends from the stem second portion 714 to the stem intermediate portion shoulder 728. The stem second portion 724 tapers from the stem intermediate portion shoulder 728 toward the stem second end 616. The stem intermediate portion 726 has a stem intermediate portion diameter 732. The stem second portion 616 has a stem second portion diameter 725. The stem tapered portion 618 has a stem tapered portion diameter 619. The stem intermediate portion diameter 732 is greater than the stem second portion diameter 725 and the stem tapered portion diameter 619. The stem intermediate portion shoulder 728 provides a mechanical stop to prevent the adapter 606 from being advanced toward the second end, beyond the stem tapered portion 618 and the stem intermediate portion shoulder 728.

The stem projection 730 extends from the stem tapered portion 618, away from the lengthwise axis 602, and is disposed adjacent to the stem intermediate portion shoulder 728. The stem projection 730 is disposed within the first recess 736 of the adapter 606, as described in more detail herein. When disposed within the first recess 736, the stem projection 730 prevents the adapter 606 from rotating relative to the stem 604. This is particularly useful when torquing an attachment member relative to the other components of an orthopedic device. The plurality of stem protrusions 690 is disposed on the stem intermediate portion 726.

In the illustrated embodiment, the adapter 606 has an adapter first end 624, an adapter second end 626, a first keel 704, a second keel 706, a first recess 736, and a second recess 738.

The first recess 736 and the second recess 738 are disposed on an axis 737 that is disposed on a plane 739 that contains the lengthwise axis 602. The first recess 736 extends from the adapter second end 626, toward the adapter first end 624, and receives the stem projection 730. The second recess 738 extends from the adapter first end 624, toward the adapter second end 626, and receives the projection 742 of the main body 610. When assembled, the first recess 736 receives the stem projection 730 and prevents the adapter 606 from rotating relative to the stem 604. When assembled, the second recess 738 receives the projection 741 and prevents the main body 610 from rotating relative to the stem 604. This is particularly useful when torquing an attachment member relative to the other components of an orthopedic device.

In the illustrated embodiment, the main body 610 comprises a single, unitary component that combines the features of the first portion 111 and the second portion 113 of a main body, as described herein. Thus, the main body 610 has a first end 638, a second end 640, a central axis 642, a passageway 646, an elongate portion 650, a protrusion 652, a wall 658, a recess 660, a notch 668, and a projection 742.

The protrusion 652 extends from the elongate portion 650 and away from the first end 638. The projection 742 extends from the protrusion 652, from the elongate portion 650, away from the first end 638, and away from the central axis 642. However, in alternative embodiments, a projection can extend only from a protrusion or an elongate portion. When assembled, the projection 742 is disposed within the second recess 738 and prevents the main body 610 from rotating relative to the adapter 606.

The attachment member 612 is disposed through the passageway 646 such that the attachment member head 670 is partially disposed within the protrusion 652. In the illustrated embodiment, the attachment member 612 has an attachment member head 670. The attachment member head 670 has an attachment member head second end 744, an attachment member first end 746, and an attachment member head intermediate portion 748 between the attachment member head second end 744 and the attachment member head first end 746. In the embodiment shown, the attachment member head 670 tapers from the attachment member head first end 746 to the attachment member head intermediate portion 748, such that the attachment member head intermediate portion 748 has a diameter 750 that is less than a diameter 752 of the attachment member head first end 746.

In the illustrated embodiment, the compression ring 720 is disposed within the protrusion 652 and between the main body 610 and the attachment member 612 (e.g., attachment member head 670). The compression plug 722 includes a compression plug recess 756 and a compression plug thread 758 that extends about a circumference of the compression plug 722. The compression plug 722 is disposed within a compression plug passageway 666. After the compression ring 720 and the attachment member 612 are installed, the compression plug thread 758 mates with a thread 759 of the main body 610 to secure the compression plug 722 to the main body 610. The compression plug 722 prevents the attachment member 612 from backing out of the stem recess 620 after implantation.

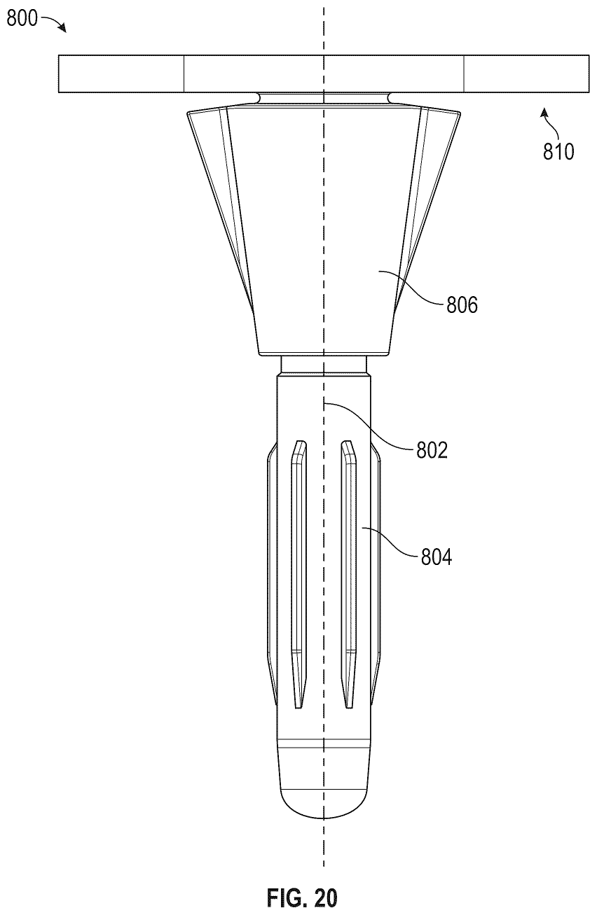

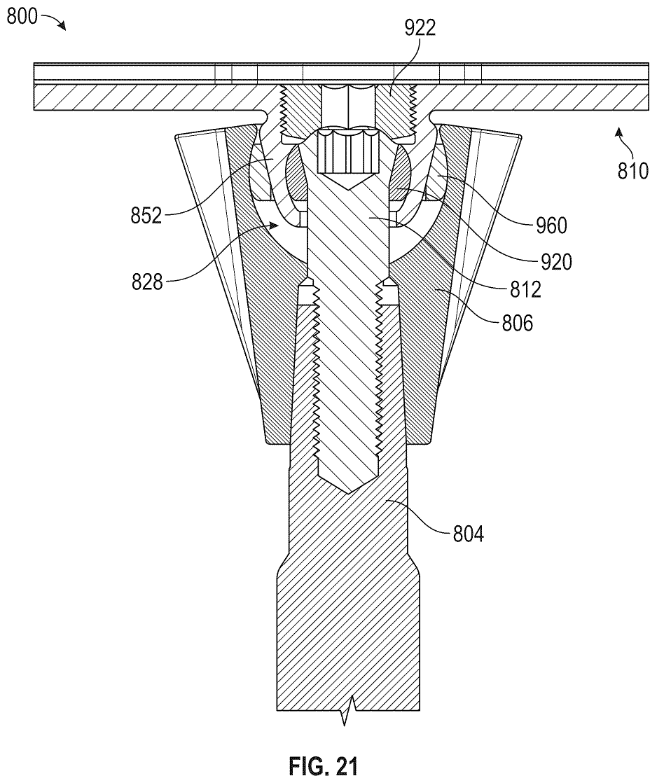

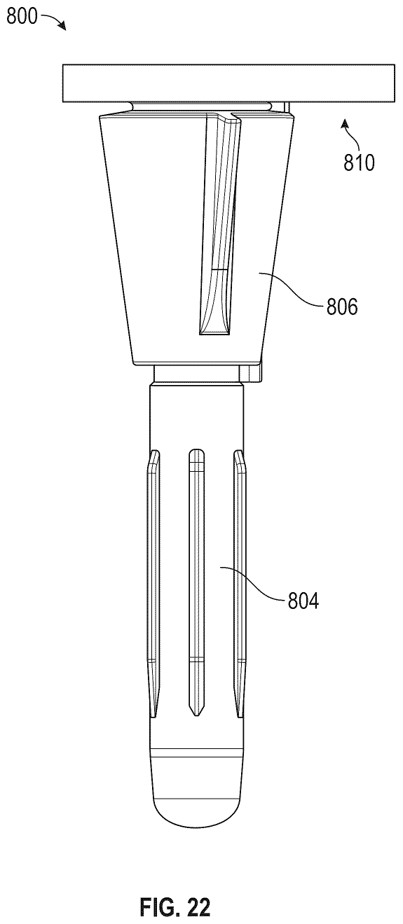

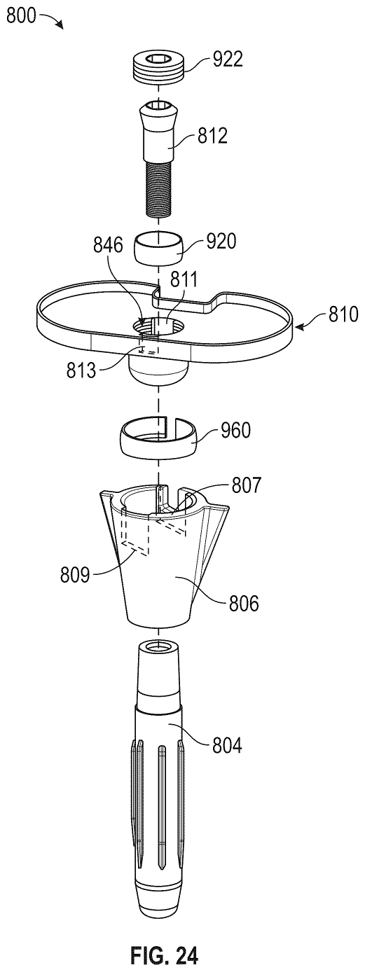

FIGS. 20 through 24 illustrate another example orthopedic device 800. The orthopedic device 800 is similar to the orthopedic devices described above, except as detailed below.

In the illustrated embodiment, the orthopedic device 800 has a lengthwise axis 802, a stem 804, an adapter 806, a main body 810, an attachment member 812, a first compression ring 920, a second compression ring 960, and a compression plug 922.

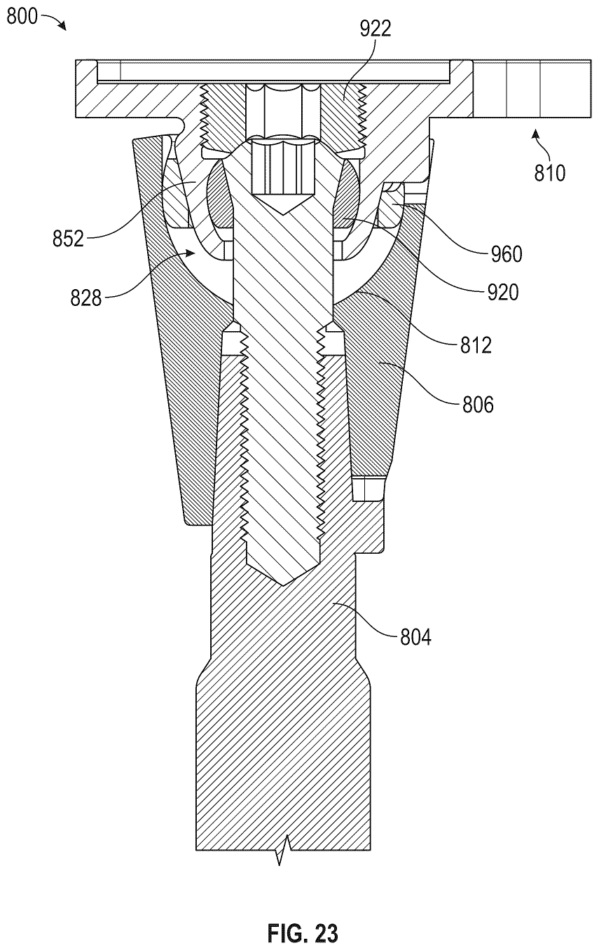

In the embodiment shown, the main body 810 includes a first main body indentation 811 and a second main body indentation 813. The first main body indentation 811 and the second main body indentation 813 are opposably facing each other and within a passageway 846 of the main body 810. During installation of the first compression ring 920, the first main body indentation 811 and the second main body indentation 813 allow the first compression ring 920 to be vertically inserted within each of the first main body indentation 811 and the second main body indentation 813. The first compression ring 920 is then rotated to be in a horizontal position, such as illustrated in FIG. 23, to be installed within the passageway 846. The first compression ring 920 is the same as the compression ring 720 described above in relation to FIGS. 15 through 19b.

The adapter 806 includes a first adapter indentation 807 and a second adapter indentation 809. The first adapter indentation 807 and the second adapter indentation 809 are opposably facing each other. During installation of the second compression ring 960, the first adapter indentation 807 and the second adapter indentation 809 allow the second compression ring 960 to be vertically inserted within each of the first adapter indentation 807 and the second adapter indentation 809. The second compression ring 960 is then rotated to be in a horizontal position, such as illustrated in FIG. 23, to be installed within the adapter recess 828.

When the orthopedic device 800 is assembled, the second compression ring 960 is positioned within an adapter recess 828. Subsequently, the main body 810 is positioned within the adapter recess 828. As a result, the second compression ring 960 is disposed within the adapter recess 828 and between the adapter 806 and the main body 810 (e.g., protrusion 852).

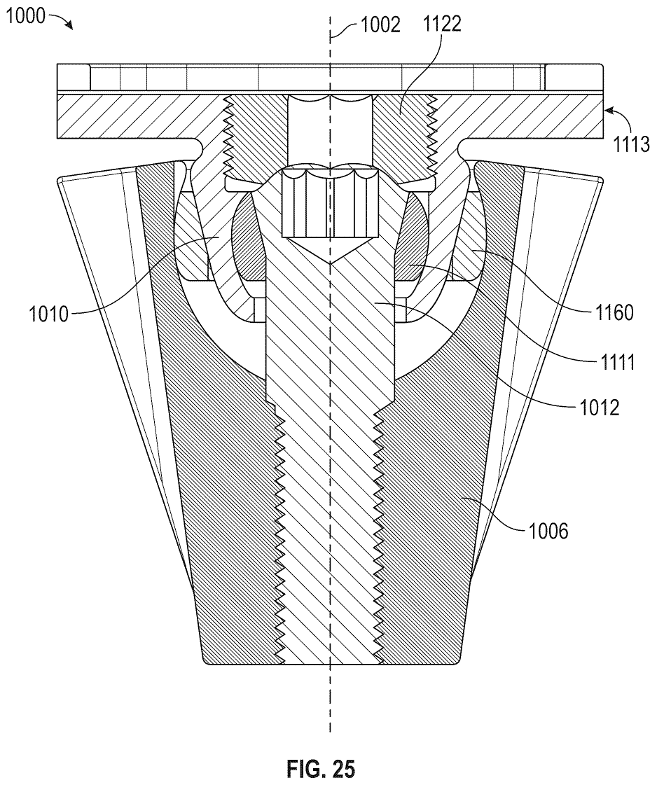

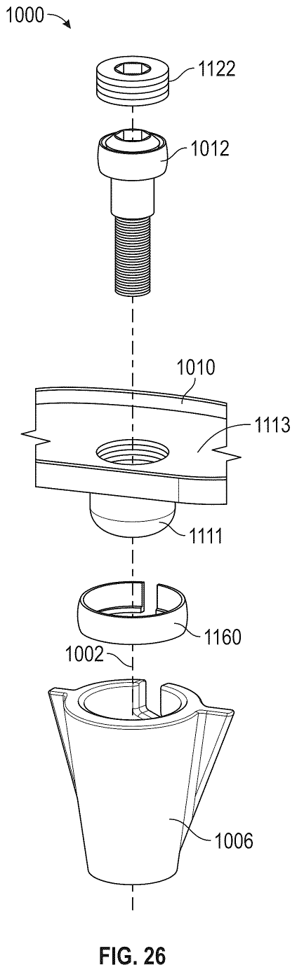

FIGS. 25 through 26 illustrate another example orthopedic device 1000. The orthopedic device 1000 is similar to the orthopedic devices described above, except as detailed below.

In the illustrated embodiment, the orthopedic device 1000 has a lengthwise axis 1002, an adapter 1006, a main body 1010, an attachment member 1012, a compression ring 1160, and a compression plug 1122.

In the embodiment shown, the orthopedic device 1000 omits the inclusion of a stem. Further, the main body 1010 has a first portion 1111 and a second portion 1113. In the embodiment shown, the second portion 1113 is not a plate. Rather, the second portion 1113 is a rectangular structure that is configured to be secured to a bone by any suitable means. Although the second portion 1113 is illustrated and described as a rectangular structure, a second portion can have any desired shape including, but not limited to, cylindrical, and any other shape considered suitable for a particular embodiment. Additionally, the second portion 1113 omits the inclusion of a notch. The compression ring 1160 is the same as the second compression ring 960 described above with respect to FIGS. 20 through 24.

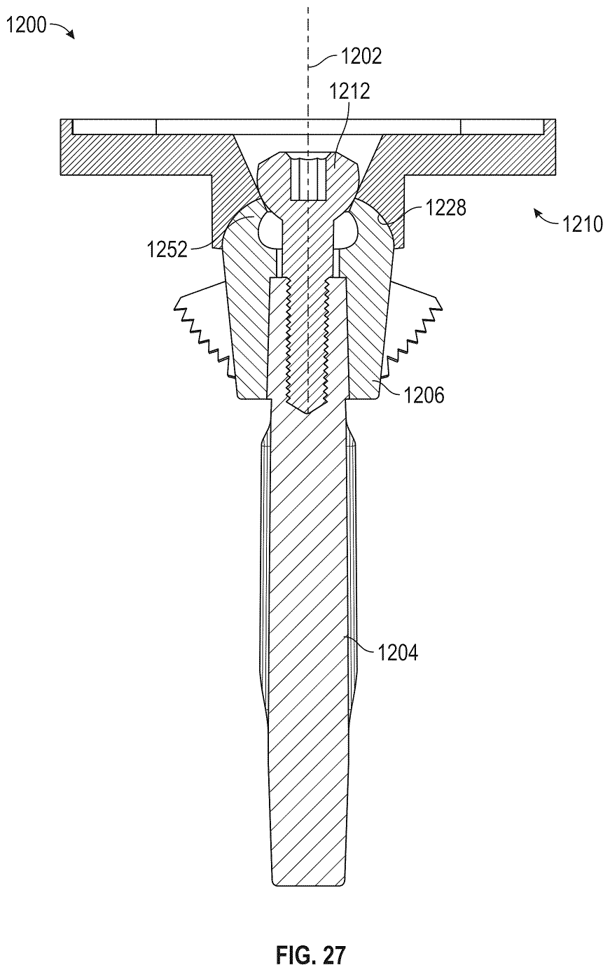

FIG. 27 illustrates another example orthopedic device 1200. The orthopedic device 1200 is similar to the orthopedic devices described above, except as detailed below.

In the illustrated embodiment, the orthopedic device 1200 has a lengthwise axis 1202, a stem 1204, an adapter 1206, a main body 1210, and an attachment member 1212.

In the illustrated embodiment, the spherical modular junction between the adapter 1206 and the main body 1210 is reversed such that the adapter 1206 functions as the protrusion 1252 and is disposed within a main body second end recess 1228 that is defined by the main body 1210. The spherical modular junction functions the same as described in previous embodiments of example orthopedic devices.

Various methods of use of an orthopedic device, such as those described herein, are described. While the methods described herein are shown and described as a series of acts, it is to be understood and appreciated that the methods are not limited by the order of acts, as some acts may in accordance with these methods may be omitted, occur in the order shown or described, occur in different orders, be repeated, or occur concurrently with other acts described herein.

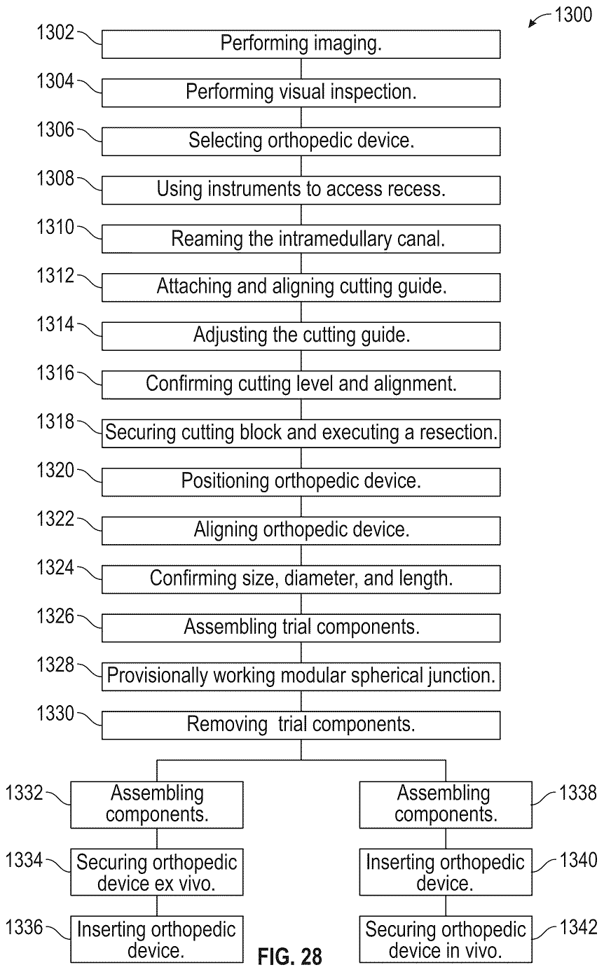

FIG. 28 illustrates a schematic of a method 1300 of using an example orthopedic device, such as those described herein.

A step 1302 comprises performing imaging to assess bone quality, identifying defects, and planning for appropriate sizing and positioning of components of the orthopedic device. Another step 1304 comprises performing visual inspection to assess bone quality, identify defects, and planning for appropriate sizing and positioning of the components. Another step 1306 comprises selecting the orthopedic device based on bone stock and alignment goals. Another step 1308 comprises using a starter awl, drill, or other instrument(s) to access a recess within the bone, such as the intramedullary canal. Another step 1310 may comprise reaming the intramedullary canal or bone, increasing diameter progressively, to achieve adequate cortical engagement and stability. Another step 1312 may comprise attaching and aligning an intramedullary or extramedullary cutting guide. Another step 1314 may comprise adjusting the intramedullary or extramedullary cutting guide for correct bone alignment. Another step 1316 may comprise confirming cutting level and alignment with a stylus or alignment guides. Another step 1318 comprises securing a cutting block and executing a resection. Another step 1320 comprises positioning an orthopedic device on the bone, with or without some sort of extension that enters the bone. Another step 1322 comprises aligning the orthopedic device and any extension with anatomic landmarks where appropriate. Another step 1324 comprises confirming size, diameter, and length of any device extensions based on previously prepared canal or bone dimensions. Another step 1326 may comprise assembling trial components of the size most appropriate for the anatomy at hand, with the modular spherical junction assembled but loose, allowing for motion, and applying the loosely assembled trial device to the prepared bone surface, and allowing any device extensions to match the natural anatomy of the patient, while a condylar side of the device is able to independently match the bone surface. For example, in the case of the proximal tibial component of a total knee device, the extension, such as a keel or stem, would enter the bone, with the modular spherical junction assembled loosely, and the device is inserted fully, allowing the proximal baseplate to sit flush on the proximal tibial plateau surface, regardless of the angle between the long axis of the tibia and the tibial plateau surface. Another step 1328 comprises provisionally locking the modular spherical junction with the attachment member such that the exact angle dictated by that individual patient's anatomy is matched and the device is fixed in that position. Another step 1330 may comprise removing the provisionally fixed trial components from the patient.

Following step 1330, step 1332 or step 1338 can be performed.

Another step 1332 comprises assembling components of the orthopedic device, such as those described herein, to arrive at an assembled orthopedic device that meets the size, shape, and orientation of the trial components. Another step 1334 comprises securing the orthopedic device in an orientation identical to that of the trial components by adjusting the orthopedic device into a locked configuration, ex vivo, such that the main body of the orthopedic device cannot be moved relative to the adapter. Another step 1336 comprises inserting the orthopedic device into the bone for final implantation.

Another step 1338 comprises assembling components of the orthopedic device, such as those described herein, to arrive at an assembled orthopedic device that meets the size and shape of the trial components. Another step 1340 comprises inserting the orthopedic device into the bone for final implementation. Another step 1342 comprises securing the orthopedic device in an orientation identical to that of the trial components by adjusting the orthopedic device into a locked configuration, in vivo, such that the main body of the orthopedic device cannot be moved relative to the adapter.

The above embodiments are to be understood as illustrative examples. Those with ordinary skill in the art will appreciate that various modifications and alternatives for the described and illustrated examples can be developed in light of the overall teachings of the disclosure, and that the various elements and features of one example described and illustrated herein can be combined with various elements and features of another example without departing from the scope of the invention. For example, it is to be understood that any feature described in relation to any one embodiment may be used alone, or in combination with other features described, and may also be used in combination with one or more features of any other of the embodiments, or any combination of any other of the embodiments. In a specific example, it is to be understood that any feature described in relation to any one embodiment of an orthopedic device may be used alone, or in combination with other features described relative to any other orthopedic device. Accordingly, the particular arrangement of elements and steps disclosed herein have been selected by the inventor(s) simply to describe and illustrate examples of the invention and are not intended to limit the scope of the invention or its protection, which is to be given the full breadth of the appended claims and any and all equivalents thereof.

1. An orthopedic device having a lengthwise axis and comprising:
an adapter having an adapter first end, an adapter second end, and an adapter recess, the adapter recess extending into the adapter from the adapter first end toward the adapter second end, the adapter recess having a first semi-spherical shape;
a main body configured to be partially disposed within the adapter recess, the main body having a first end, a second end, a central axis, and defining a protrusion, the protrusion configured to be partially disposed within the adapter recess, the protrusion having a second semi-spherical shape allowing movement of the main body along more than one axis relative to the adapter when the main body is partially disposed within the adapter recess; and
an attachment member configured to attach the main body to the adapter.

2. The orthopedic device of claim 1, wherein the adapter has an adapter second end recess;
wherein the adapter has an adapter passageway extending between the adapter recess and the adapter second end recess.

3. The orthopedic device of claim 2, wherein the main body has a passageway.

4. The orthopedic device of claim 3, further comprising a stem;
wherein the stem is attached to the adapter;
wherein the stem has a stem recess; and
wherein the attachment member is disposed through the passageway of the main body, through the adapter passageway, and into the stem recess.

5. The orthopedic device of claim 3, wherein the passageway has a conical taper.

6. The orthopedic device of claim 1, wherein the adapter further comprises an adapter attachment member extending from the adapter second end and away from the adapter first end.

7. The orthopedic device of claim 6, further comprising a stem;
wherein the stem is attached to the adapter;
wherein the stem further comprises a stem flange; and
wherein the adapter second end is disposed within the stem flange.

8. The orthopedic device of claim 7, wherein the adapter attachment member is disposed within the stem recess.

9. The orthopedic device of claim 7, wherein the stem flange has a stem flange first end and a stem flange second end;
wherein the stem flange first end defines a first flange width and the stem flange second end defines a second flange width; and
wherein the second flange width is less than the first flange width.

10. The orthopedic device of claim 1, further comprising a stem;
wherein the stem is attached to the adapter; and
wherein a portion of the stem is disposed within the adapter.

11. The orthopedic device of claim 1, further comprising a stem having a stem first end and a stem second end;
wherein the stem is attached to the adapter; and
wherein the stem first end is tapered.

12. The orthopedic device of claim 1, wherein the adapter second end is tapered.

13. The orthopedic device of claim 1, wherein the main body has a recess; and
wherein the main body comprises a first portion and a second portion; and
wherein the second portion is partially disposed in the recess.

14. The orthopedic device of claim 13, wherein the second portion is fully disposed within the recess.

15. The orthopedic device of claim 1, wherein the adapter further comprises a first keel; and
wherein the first keel extends away from said lengthwise axis.

16. The orthopedic device of claim 15, wherein the adapter first end is disposed at an angle relative to said lengthwise axis that is greater than 90 degrees.

17. The orthopedic device of claim 1, wherein the attachment member is a polyaxial screw.

18. The orthopedic device of claim 1, further comprising a stem having a plurality of stem protrusions;
wherein the stem is attached to the adapter; and
wherein the plurality of stem protrusions extend away from the stem lengthwise axis.

19. An orthopedic device having a lengthwise axis and comprising:
an adapter having an adapter first end, an adapter second end, and an adapter recess, the adapter recess extending into the adapter from the adapter first end toward the adapter second end, the adapter recess having a first semi-spherical shape, the adapter first end having a first width measured orthogonally to said lengthwise axis, the adapter second end having a second width measured orthogonally to said lengthwise axis, the second width being less than the first width;
a main body configured to be partially disposed within the adapter recess, the main body having a first end, a second end, a central axis, and defining a protrusion, the protrusion configured to be partially disposed within the adapter recess, the protrusion having a second semi-spherical shape allowing movement of the main body along more than one axis relative to the adapter when the main body is partially disposed within the adapter recess; and
an attachment member configured to attach the main body to the adapter.

20. An orthopedic device having a lengthwise axis and comprising:
a stem having a stem first end and a stem second end;
an adapter attached to the stem having an adapter first end, an adapter second end, and an adapter recess, the adapter recess extending into the adapter from the adapter first end toward the adapter second end, the adapter recess having a first semi-spherical shape, the adapter first end having a first width measured orthogonally to said lengthwise axis, the adapter second end having a second width measured orthogonally to said lengthwise axis, the second width being less than the first width;
a main body configured to be partially disposed within the adapter recess, the main body having a first end, a second end, a central axis, and defining a protrusion, the protrusion configured to be partially disposed within the adapter recess, the protrusion having a second semi-spherical shape allowing movement of the main body along more than one axis relative to the adapter when the main body is partially disposed within the adapter recess; and
an attachment member configured to attach the main body to the adapter.

21. The orthopedic device of claim 1, further comprising a compression ring;
wherein the compression ring is disposed within the protrusion and between the main body and the attachment member.
